(19) United States
(12) Patent Application Publication
(10) Pub. No.: US 2006/0160789 A1
(43) Pub. Date: Jul. 20, 2006

(54) Use of pharmacological compounds for the prevention or treatment of acute tolerance to morphines

(76) Inventors: Myriam Tirault, Sainte-Luce (FR); Bertrand Debaene, Poitiers (FR); Marie-Emmanuelle Le Guern, Compiegne (FR); Bernard Hublot, Compiegne (FR)

Correspondence Address: YOUNG & THOMPSON, 745 SOUTH 23RD STREET, 2ND FLOOR, ARLINGTON, VA, 22202, US

(21) Appl. No.: 11/320,735

(22) Filed: Dec. 30, 2005

(30) Foreign Application Priority Data
Jan. 6, 2005 (FR) 05/00112

(51) Int. Cl. A61K 31/553; A61K 31/538

(52) U.S. Cl. 514/211.09; 514/230.5

(57) ABSTRACT

The present invention relates to the use of at least one compound of the following general formula (I): 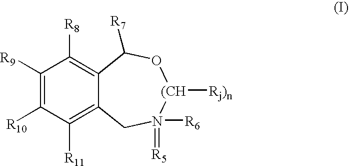 or its pharmaceutically acceptable salts, for the preparation of a medicament intended for the prevention or treatment of acute tolerance to morphines or hyperalgesia caused by morphines, in an individual having received an administration of a morphine analgesic.

The present invention relates, in particular, to the use of pharmacological compounds in order to prevent or treat acute tolerance to morphines.

Morphine and its synthetic derivatives (fentanyl, sufentanil, alfentanil, remifentanil) are powerful analgesic molecules called opioids or morphines, used in general anesthesia, in order to treat pain associated with surgery. There is great interindividual variability in the need for morphines for the same operation.

Recent scientific data suggest that the higher the dose of morphines administered during the operation, the greater the need for analgesics during the immediate post-operative period, in particular to treat the hyperalgetic effects caused by these morphines. This phenomenon is called acute tolerance to morphines.

Acute tolerance to morphines is defined by the increase in the post-operative consumption of morphine, due to the exceptional exposure to opioids during the anesthesia. It seems to appear more rapidly when the morphine is more powerful, short-acting and used in high doses (Guignard et al. (2000) Anesthesiology 93: 409-17).

Acute post-operative tolerance in particular differs from chronic tolerance, which is the reduction in analgesic efficiency of morphine over the long term, demonstrated in cancer patients with chronic pain, with correlatively, the need to increase the doses in order to obtain the same effect (Muller et al. (1999) Ann Fr Anesth Réanim 18: 866-95).

The phenomenon of acute tolerance to morphines has already been demonstrated in animals. Thus, the repeated injection of fentanyl in rats (4 doses in 1 h) leads, after intense nociceptive stimulation (pressures of increasing intensity on the rear paw), to two types of response:

early analgesic effect, with raising of the pain threshold (i.e. the possibility of applying a pressure of greater intensity, without producing a motor response in the rat),
delayed hyperalgetic effect, with reduction of the pain threshold, which effect is greater the larger the total dose of fentanyl administered.

According to the author, this sensitization to pain is due to the opioid itself, and not to a conditioning induced by the peripheral nociceptive stimulus repeated excessively; in fact, the increase in pain of the rat, after injection of a high dose of fentanyl, appears even when the rat has not been subjected to an intense nociceptive stimuli (Celerier et al. (2000) Anesthesiology 92: 465-72).

In humans, the results are comparable. In fact, in healthy volunteers, after 90 minutes of a continuous infusion of remifentanil, a marked reduction in the time during which the subject can endure the application of a thermal stimulus (cold water) or a mechanical stimulus (pressure on the non-dominant hand) (Vinik et al. (1998) Anesth Analg 86: 1307-11) is observed.

The existence of this phenomenon of acute tolerance to morphines has also been reported in a clinical situation:
after hysterectomy by laparotomy; in this case, in patients having received during anesthesia high doses of fentanyl (15 μg/kg), the quantity of morphines necessary to control the post-operative pain is increased, thus increasing the risk of the occurrence of adverse effects (respiratory depression, nausea and vomiting in particular) (Chia et al. (1999) Can J Anesth 46: 872-7).
after a laparotomy for colorectal surgery of at least two hours duration; in this case remifentanil in high doses produces an effect of acute tolerance, which, clinically, results in a greater consumption of morphine in the recovery room (Guignard et al. (2000) Anesthesiology 93: 409-17).

Thus, the use of high per-operative doses of morphines contributes to an increase in the post-operative analgesic requirements (Eisenach (2000) Anesthesiology 92: 308-9).

However, to cease using morphines in anesthesia cannot presently be envisaged, since it is essential, but instead to find the molecule which, when combined with it, prevents the phenomenon from occurring.

It has been shown that volatile halogenated agents (used for maintaining hypnosis in all previous studies), which have an anti-NMDA activity, in part reduce acute tolerance to morphines (Chauvin (2001) La Collection de la SFAR (Elsevier); Evaluation et traitement de la douleur 99-108).

Similarly, ketamine, an antagonist of the NMDA receptors, reduces, but without getting rid of, acute tolerance to alfentanil in rats (Kissin et al. (2000) Anesth Analg 91: 1483-8). It works by reducing the hyperresponsiveness of the cells of the posterior horn of the spinal cord and of glutamatergic transmission, induced by fentanyl (Celerier et al. (2000) Anesthesiology 92: 465-72).

The effectiveness of these products is limited, moreover they have undesirable effects. As a result, a subject of the present invention is to provide compounds having an effectiveness at least comparable to those already known and free of their undesirable effects in order to treat acute tolerance to morphines.

The present invention results from the inventors demonstrating, in an unexpected manner, that nefopam, a benzoxazine, allowed prevention of acute tolerance to morphines (in particular when nefopam is administered before waking a patient who has undergone a surgical operation during which he was anesthetized with doses of morphine analgesics which are standard in general anesthesia).

Thus, the present invention relates to the use of at least one compound of the following general formula (I): 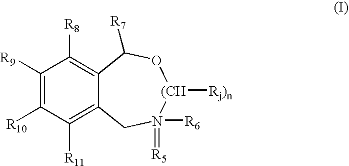
in which:
R₅ represents O or any group;
R₆ represents H or an alkyl group containing 1 to 6 carbon atoms;
n represents an integer from 2 to 4;
j represents an integer varying from 1 to n;
Rⱼ, identical or different for each substituted carbon, represents H or an alkyl group containing 1 to 6 carbon atoms;
R₇ represents a phenyl group optionally substituted by one or more identical or different groups, chosen from the list comprising H, an alkyl group containing 1 to 6 carbon atoms, an alkoxy group containing 1 to 6 carbon atoms, a trifluoromethyl group, or a halogen atom;
R₈, R₉, R₁₀, R₁₁ identical or different represent H, an alkyl group containing 1 to 6 carbon atoms, an alkoxy group containing 1 to 6 carbon atoms, a trifluoromethyl group, or a halogen atom;
   or its pharmaceutically acceptable salts,
   for the preparation of a medicament intended for the prevention or treatment of acute tolerance to morphine analgesics or hyperalgesia caused by morphine analgesics, in an individual who has received an administration of a morphine analgesic, in particular in a sufficient dose in the context of a surgical operation.

The invention relates more particularly to the use as defined above of the compounds of the following formulae (Ia) and (Ib) or their mixtures:

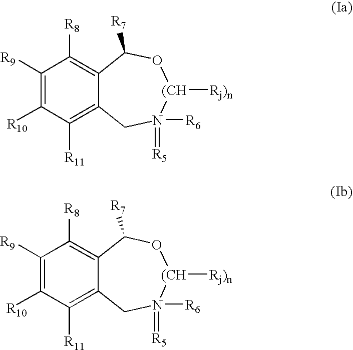

Compounds of formula (I) as well as their synthesis are in particular described in the specification of patent FR 1 590 067. The necessary adaptations to the synthesis of all of the compounds of formula (I) are easy for a person skilled in the art.

By “morphine analgesic” is meant morphine or a derivative of morphine. In particular the morphine analgesics which correspond to the following formula:

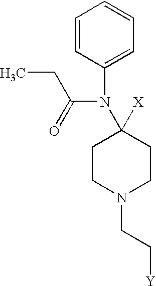

in which:
X represents H, an alkoxyalkyl group with 2 to 5 carbon atoms, or an alkoxycarbonyl group of 2 to 5 carbon atoms;
Y represents an aryl or heteroaryl group with 5 to 8 atoms optionally substituted by one or more alkyl groups with 1 to 5 carbon atoms, or an alkoxycarbonyl group with 2 to 5 carbon atoms.
By “acute tolerance to morphines” is meant the pharmacological phenomenon wherein the dose of morphine necessary to relieve post-operative pain, in particular after a surgical operation under anesthesia, is increased, as a result of the prior and exceptional exposure, during anesthesia, to morphine analgesic.
By “hyperalgesia” is meant the abnormally intense perception of a pain stimulus.

By “hyperalgesia caused by morphines” is meant the fact that exposure to morphines induces sensitivity to pain, which from a clinical point of view results in an increase in the pain scores for the same pain stimulus.

The pain scores can be measured in particular using the Visual Analogue Scale (V.A.S.) which is well known to a person skilled in the art.

Advantageously, according to the invention, the compounds of formula (I) present the property of opposing the phenomena of acute tolerance to morphines or hyperalgesia due to morphines. This property is distinct from their analgesic properties.

In one embodiment, the invention particularly relates to the use as defined above, of compounds of general formula (I), corresponding:

to nefopam of the following formula (II),

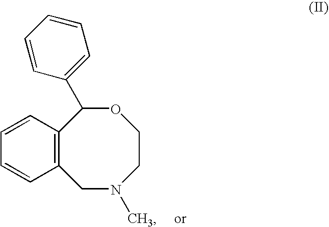

to desmethyl nefopam of the following formula (III),

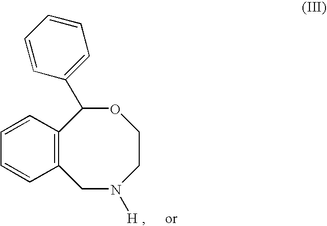

to nefopam N-oxide of the following formula (IV),

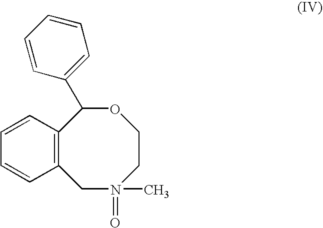

It is understood that the invention also relates to the use as defined above of the following compounds (IIa), (IIb), (IIIa), (IIIb), (IVa) and (IVb) as well as their mixtures:

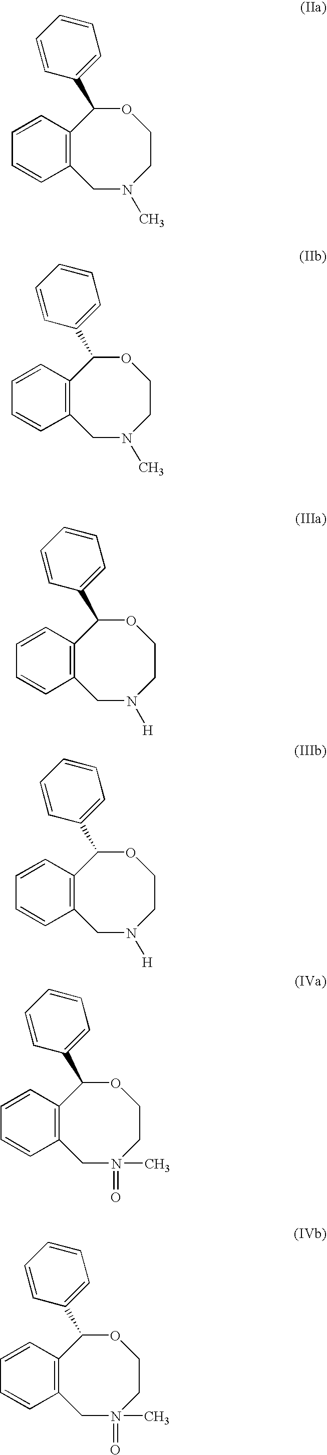

In another embodiment, the invention relates more particularly to the use as defined above, of nefopam of the following formula (II),

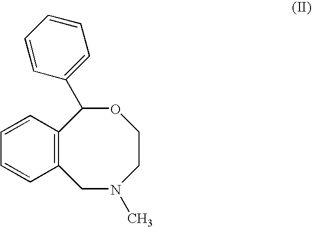

or the pharmaceutically acceptable salts of nefopam,

for the preparation of a medicament intended for the prevention or treatment of acute tolerance to morphines or hyperalgesia caused by morphines, in an individual having received an administration of a morphine analgesic.

Nefopam is the active ingredient of Acupan®. It is principally used as an analgesic during surgical operations. Moreover, this compound has the effect of saving morphine. These effects are distinct from the effects preventing the occurrence of acute tolerance to morphines or hyperalgesia caused by morphines which have been demonstrated by the inventors.

A preferred pharmaceutically acceptable salt of nefopam is nefopam hydrochloride.

In another particular embodiment of the use defined above, the medicament is suitable for the administration of a single dose of approximately 1 mg to approximately 120 mg, in particular approximately 20 mg of nefopam hydrochloride, for a dose of approximately 1 mg/day to approximately 120 mg/day, in particular approximately 20 mg/day.

According to another preferred embodiment of the use defined above, the medicament is suitable for an administration by intravenous route, by intramuscular route, or by oral route.

According to another particularly preferred embodiment of the use defined above, the morphine analgesic was administered during a surgical operation (namely in the per-operative phase), and more particularly at a sufficient conventional dose in the context of a surgical operation, in particular for a general anesthesia.

According to another particularly preferred embodiment of the use defined above, the morphine analgesic is chosen from the group comprising fentanyl, remifentanil, sufentanil, or alfentanil.

The formulae of these compounds are presented below:

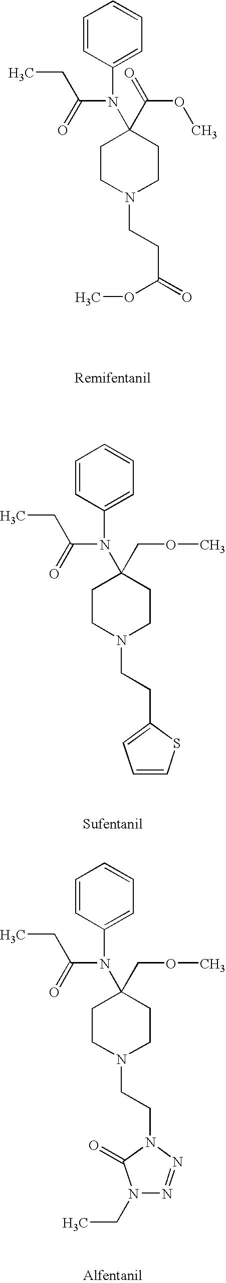

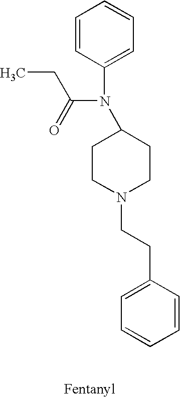

The present invention also relates to products containing:

at least one compound of formula (I) as defined above, and

at least one morphine analgesic,

as combination product for a use which is simultaneous, separate or spread over time in order to induce analgesia in a patient and in order to prevent or treat acute tolerance to morphines or hyperalgesia caused by morphines, associated with the use of said morphine analgesic in said patient.

In a particular embodiment of the products defined above, the compound of formula (I) is nefopam, desmethyl nefopam, or nefopam N-oxide, in particular nefopam.

In a yet more particular embodiment of the products defined above, the morphine analgesic is chosen from the group comprising fentanyl, remifentanil, sufentanil, or alfentanil.

In a preferred embodiment of the products defined above, the morphine analgesic is administered to the patient in order to produce an analgesic effect during a surgical operation, and the compound of formula (I), in particular nefopam, is also administered to the patient during the surgical operation, in particular before said patient wakes up (namely at the end of the per-operative phase, and before the post-operative phase).

In particular, nefopam is administered approximately 30 minutes before the patient wakes up.

### DESCRIPTION OF THE FIGURES

FIG. 1A, FIG. 1B and FIG. 1C

FIGS. 1A, 1B and 1C represent the quantity of morphine titration administered (ordinates axis, in mg/kg) to groups of patients having received a low dose (1 and 3) or a high dose (2 and 4) of remifentanyl (ordinates axis, arbitrary units) in the presence (3 and 4, phase A, black circles) or in the absence (1 and 2, phase B, white circles) of nefopam.

FIG. 1A illustrates the effect of acute tolerance to morphines; i.e. the increase in the quantity of morphine titration administered (arrow) induced by the use of a high dose of remifetanyl (2) with respect to the quantity of morphine titration administered for a low dose of remifentanyl (1, straight dotted line). In the presence of nefopam (3 and 4) the variation in the quantity of morphine titration administered is not significant.

FIG. 1B illustrates the analgesic effect of nefopam; the straight dotted line indicates what the quantity of morphine titration administered for group 4 (upper arrow) would have needed to be if nefopam only had analgesic effects.

FIG. 1C illustrates the inhibition of the effect of acute tolerance to morphines by nefopam; i.e. the reduction in the quantity of morphine titration administered for group 4 (arrow) with respect to the quantity of morphine which would have needed to be administered if nefopam had only had analgesic effects (dotted straight line).

### EXAMPLE

The study carried out by the inventors was divided into two phases:

phase A: confirmation of the phenomenon of acute tolerance to morphines,

phase B: demonstration of the inhibitory role of nefopam in this tolerance phenomenon.

1. Constitution of the Cohort of the Study

Sixty patients hospitalized for a laparotomy in the context of digestive surgery were enlisted in the study. When details and informed consent had been given, the patients were randomly separated into 2 groups for phase A (groups 1 and 2), and 2 groups for phase B (groups 3 and 4).

The patients of groups 1 and 3 received a low dose of a morphine analgesic, remifentanil, during the anesthesia (cerebral target 3 ng/ml), while the patients of groups 2 and 4 received a high dose of remifentanil (cerebral target 8 ng/ml). Moreover, the patients of groups 3 and 4 received nefopam (20 mg) before the end of the anesthesia.

The characteristics of these patients are shown in the Table 1 below:

TABLE 1
characteristics of the patients included in the study

| | Group 1 | Group 2 | Group 3 | Group 4 |
|---|---|---|---|---|
| | (n = 13) | (n = 15) | (n = 14) | (n = 15) |
| Distribution by sex (M/F) | 8/5 | 9/6 | 6/8 | 11/4 |
| Age (years) | 58 [39-68] | 55 [19-65] | 56 [43-67] | 63 [50-70] |
| Weight (kg) | 75 [48-91] | 66 [48-100] | 66 [45-108] | 77 [54-105] |
| Waist (cm) | 168 [151-178] | 172 [155-185] | 165 [151-174] | 171 [155-180] |
| BMI (cm/kg²) | 27 [20-34] | 25 [18-32] | 25 [17-35] | 27 [18-33] |
| ASA I, II, III | 2, 11, 0 | 3, 12, 0 | 3, 10, 1 | 1, 10, 4 |

BMI: body mass index
ASA: American Society of Anesthesiologists score

2. Per-Operative Period

1 h to 2 h before the start of the surgery the patients received 1 mg/kg per os of hydroxyzine (Atarax®) (usual essential treatment).

Then the patients received an anesthesia protocol using:

as muscle relaxant: atracurium

as hypnotic: propofol

as morphine analgesic: remifentanil

In order to respond in the best and quickest way to the surgical stimuli, it was chosen to deliver the hypnotic and the morphine by intravenous route, thanks to an electronic syringe driver controlled by computer software. This process is called T.C.I.A: Target-Controlled Intravenous Anesthesia. The computer integrates the pharmacokinetic models of propofol and remifentanil and calculates at each instant and according to the physical characteristics of the patient (age, sex, weight), the target plasma or cerebral concentration to be obtained for an optimum level of anesthesia, and adapts the perfusion flow rate accordingly.

This type of perfusion which is suited to the patient's actual needs for anaesthetic products requires special equipment:
Diprifusor syringe driver, for T.C.I.A of propofol (dilution 10 mg/ml): it allows a rapid adaptation of the degree of hypnosis.
Fresenius Vial syringe driver, controlled by Stanpump software on a laptop computer, for the T.C.I.A of remifentanil (dilution 50 μg/ml).

Previous studies have allowed determination of the target concentrations to be reached as a function of the progression of the anesthesia in order to obtain a given level of hypnosis, for propofol, or a given level of analgesia, for remifentanil.

| | PROPOFOL | REMIFENTANIL |
|---|---|---|
| | target plasma | target plasma |
| STAGE | concentration - μg/ml | concentration - ng/ml |
| Intubation | 4 to 8 | 4 (associated with propofol) |
| Maintenance | 3 to 6 | 3 to 12 (associated with propofol) |
| Recovery | 1 to 2 | 1.6 (spontaneous ventilation) |

Induction of Anesthesia.

propofol is flash administered for a plasma target of 6 to 8 μg/ml,

remifentanil is introduced 1 minute after the propofol for a cerebral target of 4 ng/ml,

atracurium is then introduced via an intravenous bolus (IV) of 0.5 mg/kg.

The patient is then intubated approximately 4 minutes after the administration of propofol.

Maintenance of the Anesthesia:

for all the patients: maintenance of the curarization through an IV bolus of atracurium (iterative injections of an induction half dose according to the curameter data);

for the patients of phase A:
group 1: cerebral target of remifentanil fixed at 3 ng/ml,
group 2: cerebral target of remifentanil fixed at 8 ng/ml,

for the patients of phase B:
group 3: cerebral target of remifentanil fixed at 3 ng/ml,
group 4: cerebral target of remifentanil fixed at 8 ng/ml,

The depth of anesthesia is adjusted by modulating the administration of propofol according to the hemodynamic response to surgery (variable plasma target of 3 to 8 μg/ml).

End of the Anesthesia.

A further per-operative analgesic is carried out at the start of the closure of the peritoneum to prevent post-operative pain:

for the patients of phase A (groups 1 and 2): morphine at 0.15 mg/kg in a direct intravenous injection (DIV)

for the patients of phase B (groups 3 and 4): morphine at 0.15 mg/kg in DIV+20 mg of nefopam hydrochloride (Acupan®) in a slow intravenous injection (SIV) over 20 min.

After dressing, the administration of remifentanil and propofol is stopped and the patient is transferred to the intensive care ward.

The characteristics of the anesthesia are shown in Table 3 below:

TABLE 3
characteristics of the anesthesia

| | Group 1 | Group 2 | Group 3 | Group 4 |
|---|---|---|---|---|
| | (n = 13) | (n = 15) | (n = 14) | (n = 15) |
| Remifentanil | 0.083 | 0.239 | 0.085 | 0.203 |
| (μg/kg/min) | [0.056-0.116] | [0.184-0.455] | [0.068-0.154] | [0.124-0.379] |
| Propofol | 0.199 | 0.138 | 0.186 | 0.131 |
| (mg/kg/min) | [0.119-0.318] | [0.110-0.194] | [0.132-0.265] | [0.011-0.221] |
| Bolus of morphine | 11 | 10 | 10 | 11 |
| (mg) | | | | |
| Duration of anesthesia | 204 | 233 | 198 | 181 |
| (min) | [160-245] | [90-367] | [90-325] | [130-357] |
| Duration of surgery | 148 | 190 | 143 | 155 |
| (min) | [105-200] | [50-325] | [21-272] | [95-317] |
| Time delay of | 33 | 36 | 27 | 27 |
| preventive analgesia | [13-42] | [20-80] | [13-58] | [20-69] |
| (min) | | | | |
| Time delay of | 34 | 25 | 39 | 24 |
| extubation (min) | [10-105] | [12-65] | [9-74] | [12-108] |
| Time delay of titration | 37 | 10 | 17 | 20 |
| (min) | [5-90] | [1-63] | [1-57] | [6-70] |

duration of the anesthesia: from the time of induction to the end of the anesthesia products;
duration of the surgery: from the time of incision to the end of dressing;
time of preventive analgesia: from the peroperative injection of the preventive bolus of
morphine to the stopping of the anesthesia products;
extubation time: from the stopping of the anesthesia to the removal of the intubation probe;
titration time: from the extubation to the first injection of morphine titration.

3. Post-Operative Period:

When he awakes, the patient is asked to grade his pain on the V.A.S scale (Visual Analogue Scale, graded from 0 to 100). He then receives repeated intravenous injections of morphine (bolus of 3 mg DIV), until his pain has eased (V.A.S≦30). This conventional procedure for the treatment of acute post-operative pain is called morphine TITRATION. It is carried out in the intensive care ward.

The patient is then provided with a morphine pump via a dedicated peripheral venous route, P.C.A (Patient Controlled Analgesia) in order to provide the analgesia during the hospitalization. It is activated by the patient himself when he feels pain again: the P.C.A pump (MASTER-PCA pump from VIAL) then automatically delivers a bolus of analgesic (1 mg), within the consumption limits programmed by the doctor. The P.C.A allows the self-management of pain by the patient, avoiding the underdosage and the overdosage of morphine. It is generally agreed that this system reduces the occurrence of adverse effects associated with the use of morphine. Morphine PCA is currently the benchmark in the treatment of acute post-operative pain, and is widely used.

The post-operative monitoring (analgesia, occurrence of adverse effects) is carried out in the intensive care ward, then in the hospital.

The results of the study are presented in Table 4 below:

TABLE 4
evaluation of post-operative pain

| | Group 1 (n = 13) | Group 2 (n = 15) | Group 3 (n = 14) | Group 4 (n = 15) |
|---|---|---|---|---|
| Morphine titration | 0.164 | 0.277 | 0.082 | 0.130 |
| (mg/kg) | [0.033-0.407] | [0.039-0.375] | [0-0.319] | [0-0.350] |
| Morphine titration | 12 | 18 | 7.5 | 9 |
| (mg) | [3-24] | [3-27] | [0-15] | [0-24] |
| Non-titrated patients | 0 | 0 | 4 | 1 |
| Morphine PCA over | 0.450 | 0.385 | 0.255 | 0.454 |
| 24 h (mg/kg) | [0.180-0.770] | [0.050-1.010] | [0-1.492] | [0.095-0.871] |
| Total morphine over | 0.573 | 0.661 | 0.365 | 0.649 |
| 24 h (mg/kg) | [0.310-0.984] | [0.150-1.340] | [0-1.736] | [0.095-1.210] |

4. Analysis of the Results

The principal criterion for judgment is the dose of morphine titration. The results of the study were subjected to a Mann-Whitney test and an event was considered significant if p≦0.05 with a risk a of 5% and a power of 80%.

The comparison of groups 1 and 2 indicates that the use of remifentanil in a high dose (8 ng/ml) during the anesthesia induces a statistically significant over-consumption of morphine titration of the order of 68% compared to a use of remifentanil in a low dose (3 ng/ml).

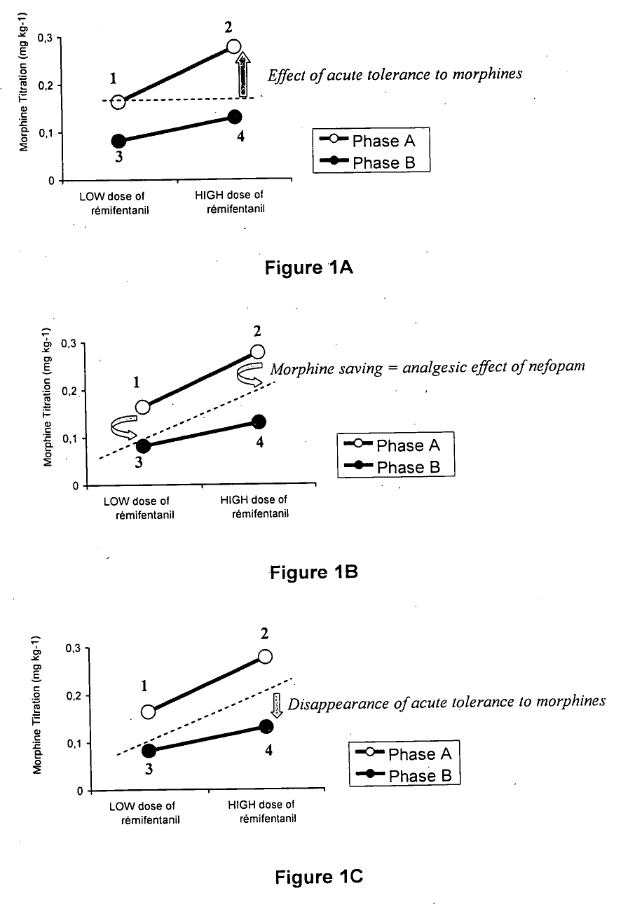

This therefore confirms the phenomenon of acute tolerance to morphine analgesics (FIG. 1A).

Moreover, the comparison of groups 3 and 4 indicates that the per-operative administration of 20 mg of nefopam hydrochloride reduces the over-consumption of morphine titration of the group which has received a high dose of remifentanil compared to the group which received a low dose thereof until it becomes statistically non-significant (FIG. 1A).

This implies therefore that the administration of nefopam makes it possible to inhibit the occurrence of the phenomenon of acute tolerance to morphine analgesics.

Moreover, FIG. 1C, which uses the above results, indicates that the quantity of morphine titration administered for group 4 is smaller than that which would have had to be administered to the group if nefopam only had analgesic effects (FIG. 1B).

This therefore clearly demonstrates that the inhibiting effect of nefopam on acute tolerance to morphine is distinct from its analgesic effect.

1-11. (canceled)

12. A method for the prevention or treatment of acute tolerance to morphines or hyperalgesia caused by morphines, in an individual who has received an administration of a morphine analgesic, comprising the administration of a pharmaceutically acceptable amount of at least one compound of the following general formula (I): 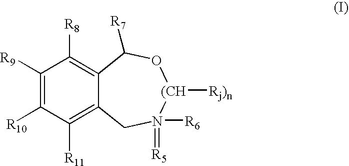
in which:
R₅ represents 0 or any group;
R₆ represents H or an alkyl group containing 1 to 6 carbon atoms;
n represents an integer from 2 to 4;
j represents an integer varying from 1 to n;
Rⱼ, identical or different for each substituted carbon, represents H or an alkyl group containing 1 to 6 carbon atoms;
R₇ represents a phenyl group optionally substituted by one or more identical or different groups, chosen from the list comprising H, an alkyl group containing 1 to 6 carbon atoms, an alkoxy group containing 1 to 6 carbon atoms, a trifluoromethyl group, or a halogen atom;
R₈, R₉, R₁₀, R₁₁ identical or different represent H, an alkyl group containing 1 to 6 carbon atoms, an alkoxy group containing 1 to 6 carbon atoms, a trifluoromethyl group, or a halogen atom;
or of its pharmaceutically acceptable salts.

13. The method according to claim 12, comprising the administration of compounds of general formula (I), corresponding:
to nefopam of the following formula (II), 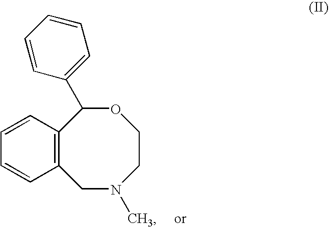
to desmethyl nefopam of the following formula (III), 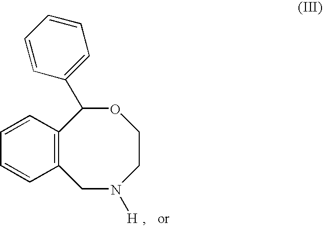
to nefopam N-oxide of the following formula (IV),

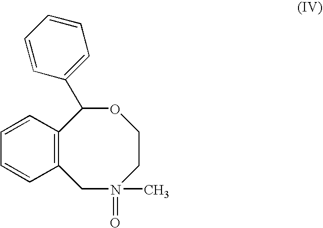

14. The method according to claim 12, comprising the administration of nefopam of the following formula (II), 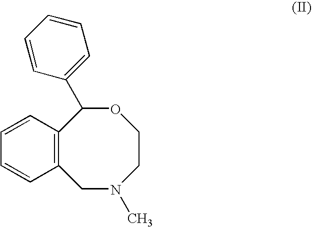
or of the pharmaceutically acceptable salts of nefopam.

15. The method according to claim 12, characterized in that the compound of the general formula (I) or its pharmaceutically acceptable salts is suitable for the administration of a single dose of approximately 1 mg to approximately 120 mg of nefopam hydrochloride, for a dosage of approximately 1 mg/day to approximately 120 mg/day.

16. The method according to claim 12, characterized in that the compound of the general formula (I) or its pharmaceutically acceptable salts is suitable for an administration by intravenous route, by intramuscular route, or by oral route.

17. The method according to claim 12, characterized in that the morphine analgesic was administered during a surgical operation.

18. The method according to claim 12, characterized in that the morphine analgesic is chosen from the group comprising fentanyl, remifentanil, sufentanil, or alfentanil.

19. Products containing:
at least one compound of the following general formula (I): 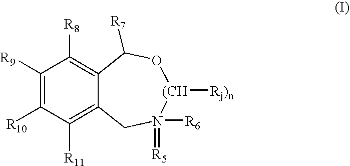
in which:
R₅ represents O or any group;
R₆ represents H or an alkyl group containing 1 to 6 carbon atoms;
n represents an integer from 2 to 4;
j represents an integer varying from 1 to n;
Rⱼ, identical or different for each substituted carbon, represents H or an alkyl group containing 1 to 6 carbon atoms;
R₇ represents a phenyl group optionally substituted by one or more identical or different groups, chosen from the list comprising H, an alkyl group containing 1 to 6 carbon atoms, an alkoxy group containing 1 to 6 carbon atoms, a trifluoromethyl group, or a halogen atom;
R₈, R₉, R₁₀, R₁₁ identical or different represent H, an alkyl group containing 1 to 6 carbon atoms, an alkoxy group containing 1 to 6 carbon atoms, a trifluoromethyl group, or a halogen atom;
or of its pharmaceutically acceptable salts, and
at least one morphine analgesic,
as a combination product for a use which is simultaneous, separate or spread over time in order to induce analgesia in a patient and in order to prevent or treat acute tolerance to morphines or hyperalgesia caused by morphines, associated with the use of said morphine analgesic in said patient.

20. Products according to claim 19, in which the compound of formula (I) is nefopam, desmethyl nefopam, or nefopam N-oxide.

21. Products according to claim 19, in which the morphine analgesic is chosen from the group comprising fentanyl, remifentanil, sufentanil, or alfentanil.

22. Products according to claim 19, in which the morphine analgesic is administered to the patient in order to produce an analgesic effect during a surgical operation, and the compound of formula (I) is also administered to the patient during the surgical operation, in particular before said patient wakes up.
